(19) United States
(12) Patent Application Publication
(10) Pub. No.: US 2005/0081282 A1
(43) Pub. Date: Apr. 21, 2005

(54) Goggle

(76) Inventor: Neng-Chang Yu, Hsinchu Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/687,651

(22) Filed: Oct. 20, 2003

(51) Int. Cl. A61F 9/02

(52) U.S. Cl. 2/428

(57) ABSTRACT

A goggle includes a body, the body having two integrated frames respectively extended around the lenses thereof and two lugs respectively outwardly extended from the frames for the mounting of a headband, each frame each having a smoothly curved inner edge fitting the curvature of human face around the eyes and a stepped inner section, and two sealing members directly molded from a compound rubber on the body, each sealing member having two annular flanges respectively bonded to the inner and outer surfaces of the corresponding frame, the outer annular flange being bonded to the outer surface of the stepped inner section of the corresponding frame and disposed in flush with an outer surface of the corresponding frame.

### BACKGROUND OF THE INVENTION

a. Field of the Invention

The present invention relates to a goggle and, more particularly, to a durable watertight goggle.

b. Description of the Related Art

A goggle is an important device for the athletic game of swimming. A goggle generally comprises a pair of lenses, a sealing frame holding the lenses, and a headband adapted to secure the frame to the user's head. When fastened to the head, the sealing frame is closely attached to the skin of the face around the eyes. Because the sealing frame is to be attached to the user's skin, the factor of comfortable wearing must be taken into account. Previously, high-density foam materials were commonly used for making sealing frame for goggle. However, due to poor watertight property, these materials are abandoned.

Nowadays, TPR (thermoplastic rubber) is commonly used for making sealing frame for goggle. A TPR sealing frame can be made having a thin and flexible peripheral edge that can be closely attached to the skin of the face, eliminating leak-in of water. The connection between the sealing frame and the lenses may be achieved by either adhesion method or insertion method. The adhesion method is to join the sealing frame and the lenses together by means of the application of glue. This method increases the manufacturing cost of the goggle, complicates quality control, and cannot completely eliminate leak-in of water. The insertion method is to directly insert the lenses into the respective grooves in the sealing frame. However, the lens holes of the sealing frame may be stretched to expand during insertion of the lenses, thereby causing leak-in of water. A goggle made according to this method produces high resistance during swimming, and is less valuable.

FIGS. 1 and 2 show a goggle according to the prior art. This structure of goggle 1 comprises two lenses 11, and a sealing frame 12 directly molded from thermoplastic rubber on the lenses 11. Because the sealing frame 11 is directly molded on the lenses 11, the connection area between the border 111 of each lens 11 and the sealing frame 12 and the lenses 11 is maintained in a perfectly watertight status. However, because the sealing frame 11 is directly molded on the border of each lens 11, the transverse L1 width of the effective view angle of the lenses 12 is limited. Further, the sealing frame 12 has two through holes 121 at two sides for the mounting of the headband. When the goggle fastened to the user's head, the stretching force F from the headband forces the sealing frame 11 to expand. Frequently expanding of the sealing frame 11 may cause the connection area between the border 111 of each lens 11 and the sealing frame 12 to break.

FIGS. 3 and 4 show another structure of goggle according to the prior art. According to this design, each lens 11 has a plurality of mounting through holes 112 at the border area 111. During molding of the sealing frame 12 on the lenses 11, the thermoplastic rubber fills up the mounting through holes 112. This design of goggle is relatively more durable in use. However, the problem of limited view angle still exists.

For better refraction power and transmissivity, polycarbonate is preferably used for making lenses. However, it is difficult to mold a sealing frame from thermoplastic rubber on polycarbonate lenses. Further, a goggle having lenses of polycarbonate and a sealing frame of thermoplastic rubber is not durable in use.

### SUMMARY OF THE INVENTION

The present invention has been accomplished under the circumstances in view. It is one object of the present invention to provide a goggle, which has a wide view angle and low water resistance. It is another object of the present invention to provide a goggle, which is easy to make. It is still another object of the present invention to provide a goggle, which is durable in use. It is still another object of the present invention to provide a goggle, which gives a comfort wear.

To achieve these and other objects of the present invention, the goggle comprises a body having two lenses, two sealing members respectively bonded to the body and closely attachable to the skin of the user's face, and a headband adapted to fasten the body to the user's head; wherein the body comprises two integrated frames respectively extended around the lenses, and two lugs respectively outwardly extended from the frames for the mounting of the headband, the frames each having a smoothly curved inner edge fitting the curvature of human face around the eyes, and a stepped inner section; the sealing members are directly molded from a compound rubber on the body, each having two annular flanges respectively bonded to inner and outer surfaces of the frames, the outer annular flange being bonded to an outer surface of the stepped inner section of the corresponding frame and disposed in flush with an outer surface of the corresponding frame.

### BRIEF DESCRIPTION OF THE DRAWINGS

FIG. 1 is a perspective view of a goggle according to the prior art.

FIG. 2 is a sectional view in an enlarged scale of the goggle shown in FIG. 1.

FIG. 3 is a perspective view of another structure of goggle according to the prior art.

FIG. 4 is a cutaway of in an enlarged scale of a part of the goggle shown in FIG. 3.

FIG. 5 is a perspective view of a goggle according to the present invention.

FIG. 6 is a sectional view in an enlarged scale of a part of the goggle shown in FIG. 5, showing the structure of the body.

FIG. 7 is a schematic sectional drawing showing the sealing member bonded to the corresponding frame of the body.

FIG. 8 is a sectional view in an enlarged scale of the goggle according to the present invention.

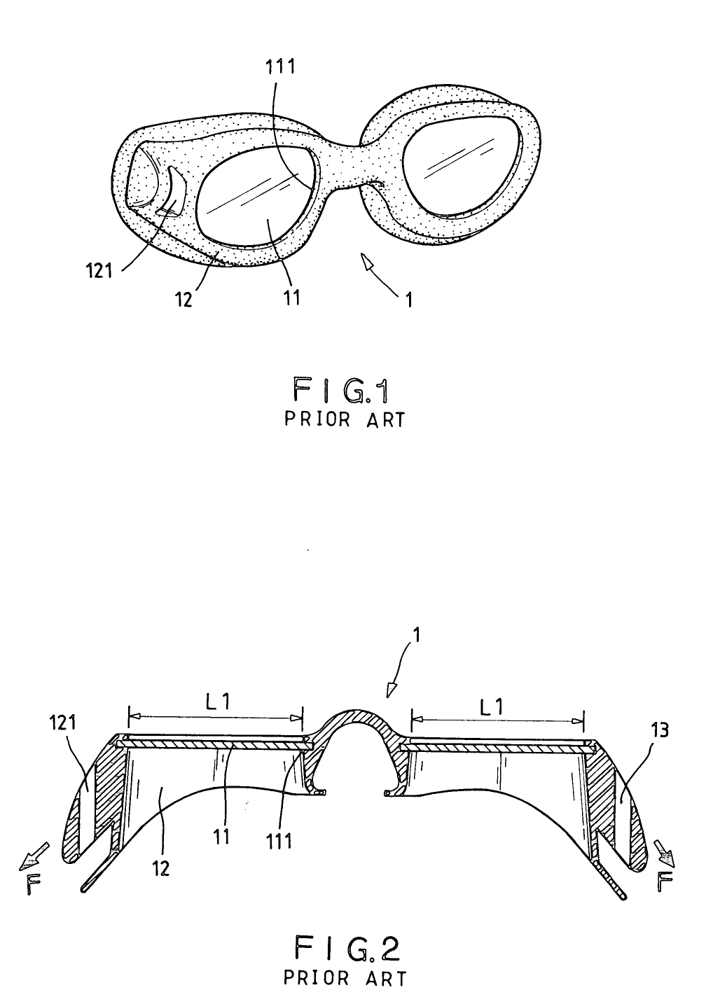

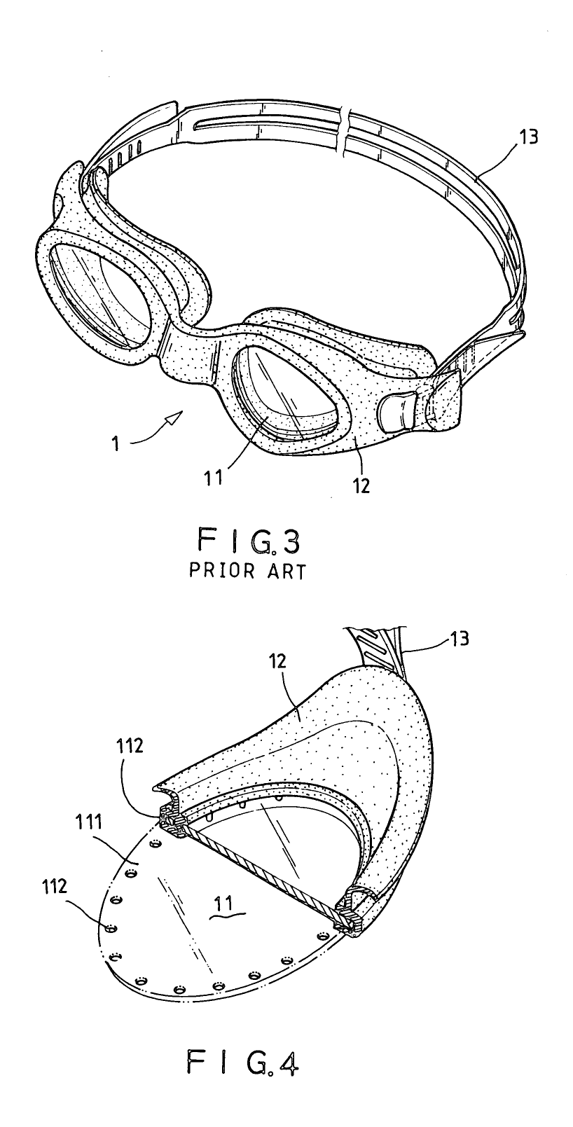

### DETAILED DESCRIPTION OF THE PREFERRED EMBODIMENTS

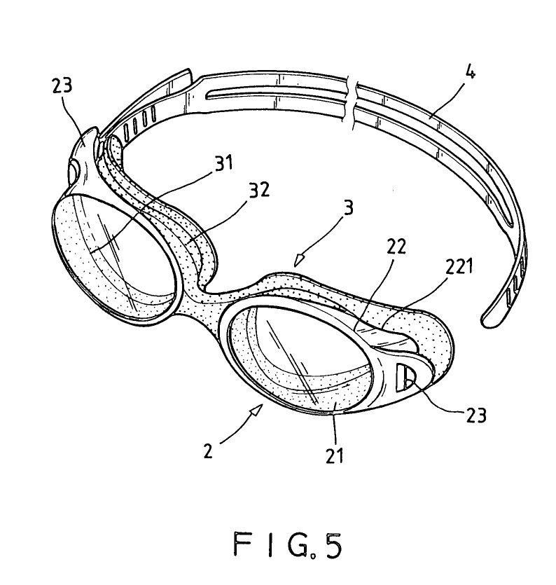

Referring to FIG. 5, a goggle in accordance with the present invention is shown comprised of a body 2, two sealing members 3 respectively molded on the body 2 from compound rubber and closely attachable to the skin of the user's face to prevent leak-in of water, and a headband 4 adapted to fasten the body 2 to the user's head.

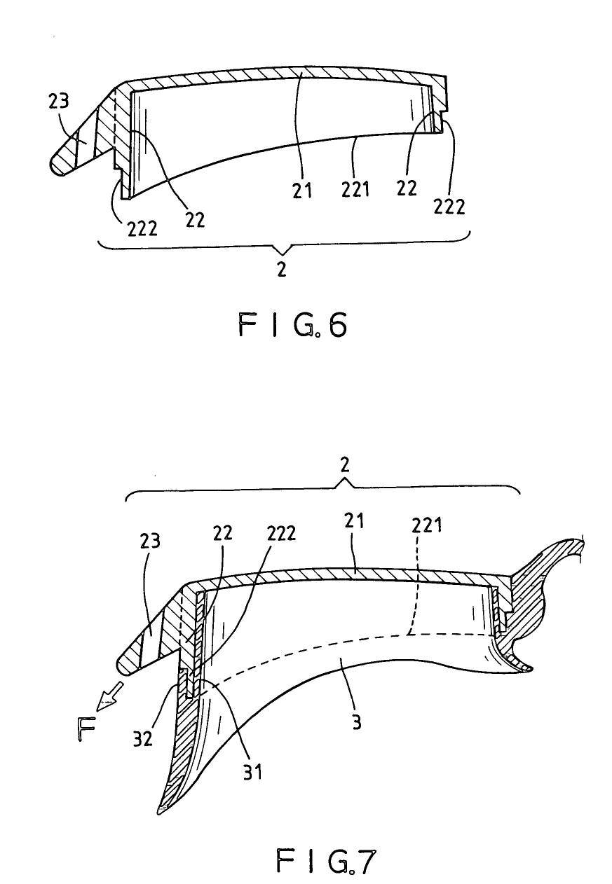

Referring to FIG. 6, the body 2 comprises two lenses 21, two integrated frames 22 respectively extended around the lenses 21, and two lugs 23 respectively outwardly extended from the frames 22 for the mounting of the headband 4. The frames 22 each have a smoothly curved inner edge 221 fitting the curvature of human face around the eyes, and a stepped inner section 222.

Referring to FIG. 7, the sealing members 3 are directly molded from compound rubber on the body 2 in the cavity of a mold (not shown). Each sealing member 3 has two annular flanges 31 and 32 respectively bonded to the inner and outer surfaces of one frame 22. The outer annular flange 32 is bonded to the outer surface of the stepped inner section 222 of the corresponding frame 22, and disposed in flush with the outer surface of the corresponding frame 22.

The body 2 is preferably molded from polycarbonate for the advantages of good refraction power and transmissivity. Further, because polycarbonate has certain strength, the body 2 does not deform easily, and the lugs 23 doe not deform and break when stretched by the headband 4.

The compound rubber for the sealing members 3 is a thermoplastic rubber added with a bonding agent. When molded on the body 2 in the cavity of the mold, the sealing members 3 does not stick to the periphery of the cavity of the mold. After molding, the sealing members 3 and the body 2 are tightly bonded together, preventing a water leakage.

The chemical properties of the compound rubber for the sealing members 3 ensure tight bonding between the body 2 and the sealing members 3. Because the annular flanges 31 and 32 of the sealing members 3 are respectively bonded to the inner and outer surfaces of frames 22, no air gap exists in between the sealing members 3 and the body 2. When fastened to the user's head, the sealing members 3 are closely attached to the skin of the user's face, working like a vacuum mount.

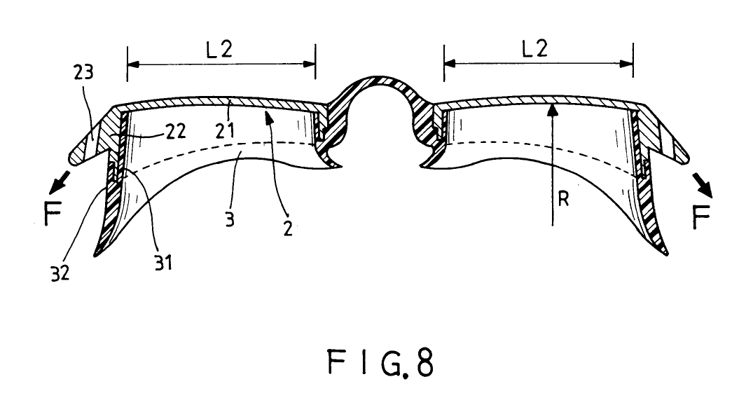

Referring to FIG. 8, because the annular flanges 31 and 32 of the sealing members 3 are respectively bonded to the inner and outer surfaces of frames 22 but not directly fastened to the border of each lens 21, the sealing members 3 do not block any part of the full view angle L2 of each lens 21. Further, the lenses 22 can be made smoothly curved outwards to increase view angle and to reduce resistance.

Because the goggle is an integrated device before the mounting of the headband 4, it has a streamline configuration without complicated fastening structure. This orthopedically engineered goggle meets fluid mechanics, and is compatible to human skin. Because the headband 4 is fastened to the lugs 23 of the body 2, stretching force F from the headband 4 is transmitted to the lugs 23 to further force the sealing members 3 against the skin of the user's face, keeping the sealing members 3 closely attached to the skin of the user's face in a watertight manner.

While only one embodiment of the present invention has been shown and described, it will be understood that various modifications and changes could be made thereunto without departing from the spirit and scope of the invention disclosed.

1. A goggle comprising a body having two lenses, two sealing members respectively bonded to said body and closely attachable to the skin of the user's face, and a headband adapted to fasten said body to the user's head;
wherein said body comprises two integrated frames respectively extended around said lenses, and two lugs respectively outwardly extended from said frames for the mounting of said headband, said frames each having a smoothly curved inner edge fitting the curvature of human face around the eyes, and a stepped inner section; said sealing members are directly molded from a compound rubber on said body, each having two annular flanges respectively bonded to inner and outer surfaces of said frames, said outer annular flange being bonded to an outer surface of the stepped inner section of the corresponding frame and disposed in flush with an outer surface of the corresponding frame.

2. The goggle as claimed in claim 1, wherein said compound rubber is composed of a thermoplastic rubber and a bonding agent.
